(19) United States
(12) Patent Application Publication
(10) Pub. No.: US 2003/0208196 A1
(43) Pub. Date: Nov. 6, 2003

(54) Control system for limited-use device

(75) Inventor: Robert T. Stone, Sunnyvale, CA (US)

Correspondence Address: ARTHROCARE CORPORATION, 680 VAQUEROS AVENUE, SUNNYVALE, CA, 94085-3523, US

(73) Assignee: ArthroCare Corporation, Sunnyvale, CA

(21) Appl. No.: 10/139,117

(22) Filed: May 3, 2002

(51) Int. Cl. A61B 18/18

(52) U.S. Cl. 606/41

(57) ABSTRACT

This relates generally to limited use devices. More particularly, the invention relates to a control system for limited use devices, particularly, medical devices and instruments which can detect utilization history and compare the history to utilization factors to disable the device.

### BACKGROUND OF THE INVENTION

Increasingly, medical practitioners are employing disposable surgical instruments that are designed to be used a limited number of times and thereafter discarded. Many disposable instruments are intended to be employed once in a single surgical operation. Disposable surgical instruments possess a number of advantages over reusable surgical devices, e.g., they can be fabricated from less expensive materials compared to reusable devices and consequently reduce the overall costs of surgery, in general, they perform optimally since they are not subject to the wear and tear of repeated use, and they minimize the risk that infectious diseases will be transmitted to other patients.

It is important, however, to recognize that disposable surgical instruments are often equipped for a single procedure, e.g., by reason of a limited number of staples or clips or are generally not designed to withstand many repeated re-sterilizations and usages. For example, surgical staplers, clip appliers, and the like, have been provided with lock-out mechanisms that preclude actuation of the devices after they have been actuated a predetermined number of times (see, e.g., U.S. Pat. No. 4,955,959). Surgical devices that include mechanisms for displaying the number of times the device has been used or the number of times the device has been heated, e.g., by autoclave sterilization, are also known (see e.g., U.S. Pat. No. 5,313,935 and EP0581400). However, the conventional methods employed to track the remaining useful lifetime of such devices consist primarily of recording in a ledger the number of operations that the device has been used, or the number of times the device has been sterilized, without regard to the actual usage of the device. Such recording systems are prone to bookkeeping errors, and may even over-estimate or underestimate the condition of the device(s).

It would thus be desirable for limited-use or disposable devices, particularly, medical instruments, to have at least one enabling means that renders the device inoperable until an enablement signal is provided.

It would further be desirable if such devices included a means for accurately tracking the usage (either the number of uses or the duration of use) of a limited-use/disposable device. The ability to track device usage increases the probability that a limited-use or disposable device is not used beyond its intended life-span and is replaced before it fails. This ability may prove to be vital for limited-use devices used in critical medical applications, such as surgical operations, or monitoring critically ill patients. Similarly, it may be extremely important to track the usage of certain devices where important components may require periodic servicing and/or component replacement.

In view of the above, there remains a need for a cost effective and efficient control system for a limited-use device having enabling means that renders the device inoperable until a signal is provided by the control system. Such a control system could be used in limited-use devices and would include a means for accurately tracking the usage of the device.

A need also remains for limited-use devices having control systems that can be readily programmed to monitor and respond to a plurality of utilization factors, including actuation events (e.g., start-ups), time or duration in use and pre-use events.

There also remains a need for control systems and/or limited-use devices incorporating the control system that are able to withstand the stringent requirements of medical devices in general (e.g., manufacturing, packing, sterilization, transport, reliability, etc.)

### SUMMARY OF THE INVENTION

A variation of the invention includes a limited-use device for use with a power supply comprising, a device body having a proximal and distal portions, at least one active component located on the distal portion, the component adapted to be activated by the power supply, a control module attached to the device body, the module adapted to store and compare at least one utilization factor and a utilization history, and being adapted to provide an enablement signal upon comparing the utilization factor and utilization history; an enablement circuit in communication between the device and the power supply, the enablement circuit including an effective SCR that is adapted to enable the device in response to the enablement current.

Another variation of the invention includes a limited-use device for use with a power supply, the power supply having an energized and de-energized state, the device comprising, a device body having a proximal and distal portions, at least one active component located on the distal portion, the component adapted to be activated by the power supply, a control module attached to the device body, the module adapted to register a utilization history based on connection of the device to the power supply when the power supply is in the energized state, the control module also adapted to provide an enablement signal upon comparing a utilization factor to the utilization history, an enablement circuit in communication between the device and the power supply, and adapted to enable the device in response to the enablement current.

A variation of the control module of the invention may comprise a memory module and a processing module, where the processing module and memory module are in communication. The memory module may be a device selected from the group consisting of electrically erasable programmable read only memory, nonvolatile random access memory, battery backed up random access memory, magnetic data storage apparatus, and optical data storage.

A variation of the invention includes memory module which stores the utilization factor and the utilization history. A variation of the invention also includes a processing module adapted for comparing the utilization factor and the utilization history.

A variation of the control system for a limited-use device in accordance with this invention comprises a control module having a memory module and a processing module, the memory module being adapted to store an execution program, a plurality of utilization factors and utilization history, the processing module being adapted to monitor the utilization history and provide an enablement current in response to the plurality of utilization factors; and an enablement circuit having an effective silicon control rectifier (SCR) that is adapted to enable the device in response to the enablement current.

Other modes for providing limited use devices are described in commonly assigned U.S. Provisional Application Serial No. ______, filed Apr. 24, 2002, entitled, LIMITED USAGE CYCLE APPARATUS, attorney docket number CB-13P and commonly assigned U.S. patent application Ser. No. ______ filed May 2, 2002 entitled LIMITED USE DEVICES AND METHODS THEREOF, attorney docket number CB-13. The entirety of both applications are hereby incorporated by reference.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a front plan view of an example of a limited-use/disposable prior to the invention described herein;

FIG. 2 is a front plan view of a variation of a limited-use/disposable according to invention;

FIG. 3 is a schematic illustration of an embodiment of a control module, according to the invention;

FIG. 4 is an example of a flow chart of a control program employing a utilization factor, according a variation of invention;

FIG. 5 is a schematic illustration of a known silicon control rectifier (SCR);

FIG. 6 is a schematic illustration of an embodiment of a control system circuit, according to the invention; and

FIG. 7 is a further schematic illustration of the control system circuit shown in FIG. 6, according to the invention.

### DETAILED DESCRIPTION OF THE PREFERRED EMBODIMENT

Before describing details of present invention, it is to be understood that this invention is not limited to particularly exemplified systems or process parameters.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include plural referents unless the content clearly dictates otherwise. Thus, for example, reference to a “memory device”, such as a flash memory device, includes two or more such memory devices.

While the principles of the present disclosure are disclosed herein in connection with a particular limited-use medical instrument, it shall be understood that the control system and principles described in detail herein are broadly applicable to a wide array of limited-use devices in a multitude of additional fields, including, for example, pharmaceutical and forensic. The inventive concept may include devices which incorporate features used or known in the art. For example, the invention concept maybe combined with such devices as commonly known RF, microwave, ultrasound, electrosurgical, etc. devices. Moreover, medical devices such as pulse-oximeters, probes, trocars, obturators, cannulas, endoscopes, vitreous cutters, catheters, laparoscopes and electrically-powered scalpels, and the like, are encompassed by this disclosure. The invention will be described, for illustrative purposes, in connection with limited-use or disposable RF devices. Such devices include, but are not limited to devices provided by ArthroCare® Corp., Sunnyvale, Calif. (discussed in more detail below.).

It is noted that the terms “limited-use” or “disposable”, as used in connection with devices is intended to include, for example, a device, instrument or component having a predetermined duration of use or useful life. The term thus includes, but is not limited to, single procedure devices (e.g., disposable surgical instruments) and devices having limited actuation events (e.g., start-up), time or duration in use and pre-use events (e.g., sterilization).

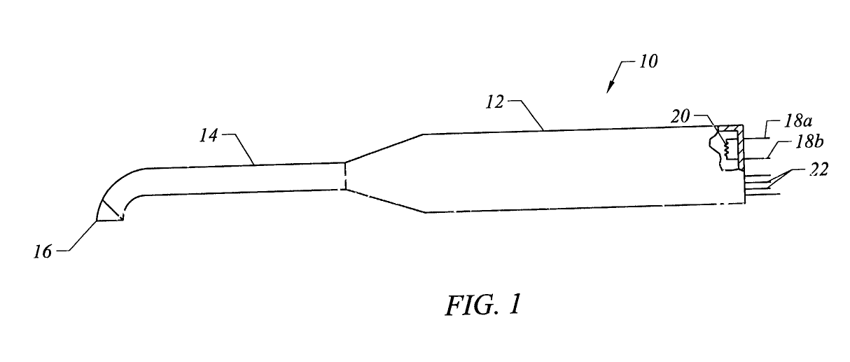

Referring first to FIG. 1, there is shown a front plan view of a RF device 10. As illustrated in FIG. 1, the device 10 includes a housing portion 12 and an ablation portion 14, extending distally from the housing portion 12.

The device 10 includes at least one electrode 22 that is adapted to transmit a predetermined level of RF energy to the ablation end 16 to apply energy to the tissue. The device 10 further includes an identity component 20 (e.g., a resistor, microchip, circuit, etc.) that is connected to power leads 18a, 18b. The identity component 20 provides a pre-set level of current and, hence, reflects the intended surgical use or procedure (e.g., pediatric tonsillectomy). In this way, a number of devices having different intended uses may be used with a single power supply. The component 20 determines the nature of the power to be applied to the device.

As will be appreciated by one having ordinary skill in the art, any number of identity components 20 may be employed to provide the desired level of current for a particular procedure.

As discussed above, the invention is intended to include electrosurgical instruments (probes or catheters) provided by ArthroCare® Corp. The use of these instruments typically involves applying a high frequency voltage between one or more active electrode(s) and one or more return electrode(s), from an electrosurgical generator, controller, or power supply, to develop high electric field intensities in the vicinity of the active electrode(s). The voltage applied between the return electrode(s) and the active electrode(s) is typically at high or radio frequency, usually between about 5 kHz and 20 MHz, and often between about 100 kHz and 200 kHz. Typical parameters of such voltages are described in commonly assigned U.S. Pat. No. 6,235,020, the disclosure of which is incorporated by reference herein in its entirety for all relevant purposes. The high electric field intensities may lead to ablation via plasma-induced molecular dissociation of tissue components. This process of volumetric removal of tissue via molecular dissociation has been termed Coblation®. A more complete description of electrosurgical instruments and methods, and the Coblation® phenomenon is provided in commonly assigned U.S. Pat. Nos. 5,683,366, 6,190,381, 6,235,020, 6,283,961, and 6,309,387, the disclosures of which are incorporated by reference herein in their entirety for all relevant purposes.

Typically, the electrosurgical generator is capable of operation in an ablation mode (for ablating tissue) or a sub-ablation mode (for coagulating or otherwise modifying the tissue). A current flow path may be provided between the active electrode(s) and the return electrode(s) by delivery of an electrically conductive fluid, as described in commonly assigned U.S. Pat. Nos. 5,697,281 and 6,312,408, the disclosures of which are incorporated by reference herein in their entirety for all relevant purposes. Additional variations of these instruments include aspiration lumen(s) and one or more aspiration electrode(s). Instruments incorporating aspiration electrode(s) are described in commonly assigned U.S. Pat. No. 6,254,600, the disclosure of which is incorporated by reference herein in its entirety for all relevant purposes. As is apparent, the aspects and features of the present invention are applicable to the above described devices.

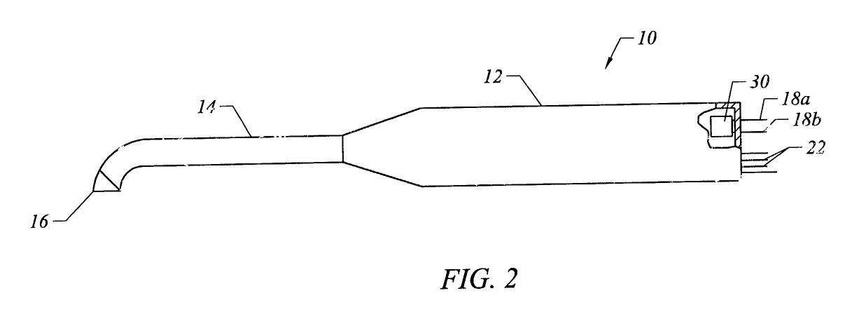

In accordance with the present invention and as shown in FIG. 2, the control system or module 30, described in detail below, may be disposed within the housing portion 12 or the connector (not shown) of the device 10. Moreover, if the device 10 includes an attached cable for coupling to an external unit, the control system or module 30 may be located therein. Preferably, the control module 30 is disposed in the housing portion (see FIG. 2) and is operatively connected to leads 18a, 18b. In any case, the module 30 will be attached to the body of the device 10 which includes the housing portion 12, the shaft carrying the active component (e.g., an electrode, the ablation end 16, transducers, etc.), and/or an external housing (not shown) attached to the device 10.

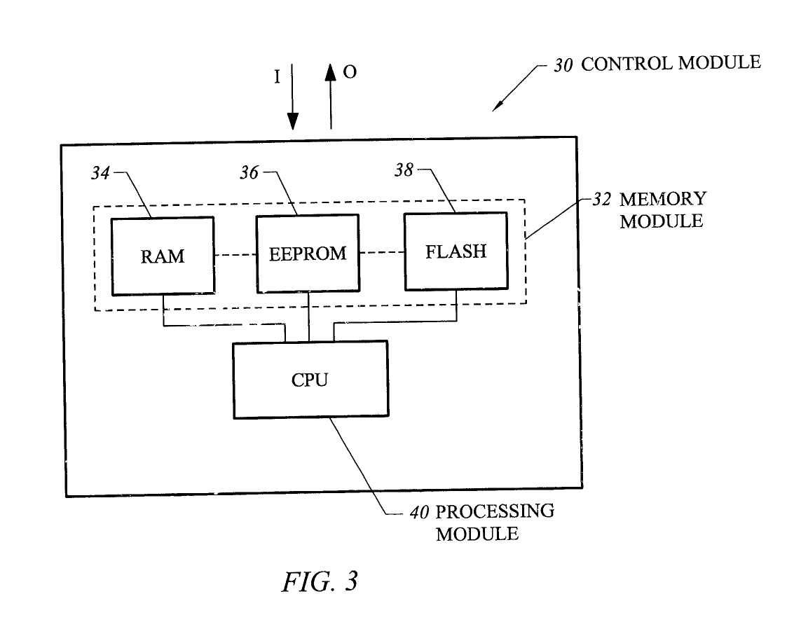

Referring now to FIG. 3, there is shown a schematic illustration of a variation of a control module 30 of the invention. As illustrated in FIG. 3, this variation of the control module 30 includes at least a memory module 32 and a processing module 40 that is in communication therewith.

According to the invention, the memory module 32 may include one or more devices that provides non-volatile memory to store various data. Such device may also be programmed during use. An example of such devices includes, but is not limited to, electrically erasable programmable read only memory (EEPROM), non-volatile RAM, battery-backed-up RAM, magnetic data storage apparatus and optical data storage apparatus, and memory devices that are not programmable during use, including, but not limited to, ROM, PROM, EPROM and flash memory.

As used herein, EEPROM is meant include any non-volatile, semiconductor memory device in which memory cells may be written to and erased on a byte-by-byte basis. The term “flash memory”, as used herein, it is meant to include any non-volatile, semiconductor memory device that is erasable in block.

As illustrated in FIG. 3, in a preferred embodiment, the memory module 32 includes RAM 34, EEPROM 36 and flash 38 memory devices. Preferably, the RAM device is employed to store temporary local variables, the EEPROM device 36 is employed to store utilization data, and the flash device is employed to store the execution or control system program.

The processing module 40 preferably comprises a microprocessor (or CPU). In one variation of the invention, the processing module 40 comprised an 8051 processor, commercially available from Atmel Corp. (San Jose, Calif.).

A variation of the invention includes preprogramming the memory module 32, or more particularly, the EEPROM device 36, with equipment utilization limits before the limited-use device is distributed by the manufacturer. An example of equipment utilization limits includes, but is not limited to, a maximum equipment actuation count, a maximum procedure count, a maximum equipment actuation time, a maximum sterilization count, and/or a maximum allowable count of connections between the device and a power supply.

In additional variations of the invention, the memory module 32 may also be preprogrammed with procedure requirements (e.g., current) and/or other data for use by the control module 30 to control the operation of a power supply module (not shown.) Such a configuration could control the power supply to provide a desired power requirements for a specified medical procedure. In the noted embodiment, when the limited-use device (e.g., device 10) is initialized, the control module 30 may initially request a transfer of preprogrammed data stored in the memory module 32. This data would then be used by the processing module 40 to regulate the power supplied by the power supply module in accordance with the transferred data. The data may include, for example, voltage ranges and limits, current ranges and limits, instrument impedance and scale factors. According to the invention, the power supply regulation may be accomplished when the memory module 32 includes any of the aforementioned memory devices, whether or not they can be programmed during use.

As discussed in detail below, in one embodiment, at least one component of the memory module 32, preferably, the EEPROM device 36, is programmable during use, and includes memory space dedicated to storing data reflecting the utilization of the limited-use device. The utilization history includes at least an accumulated equipment actuation count, accumulated procedure count or use data, accumulated equipment actuation time, and any other data deemed relevant by the manufacturer. According to the invention, the EEPROM device 36 may be programmed to update the utilization history prior to, before, during or immediately after use, as long as device, e.g., device 10, is energized.

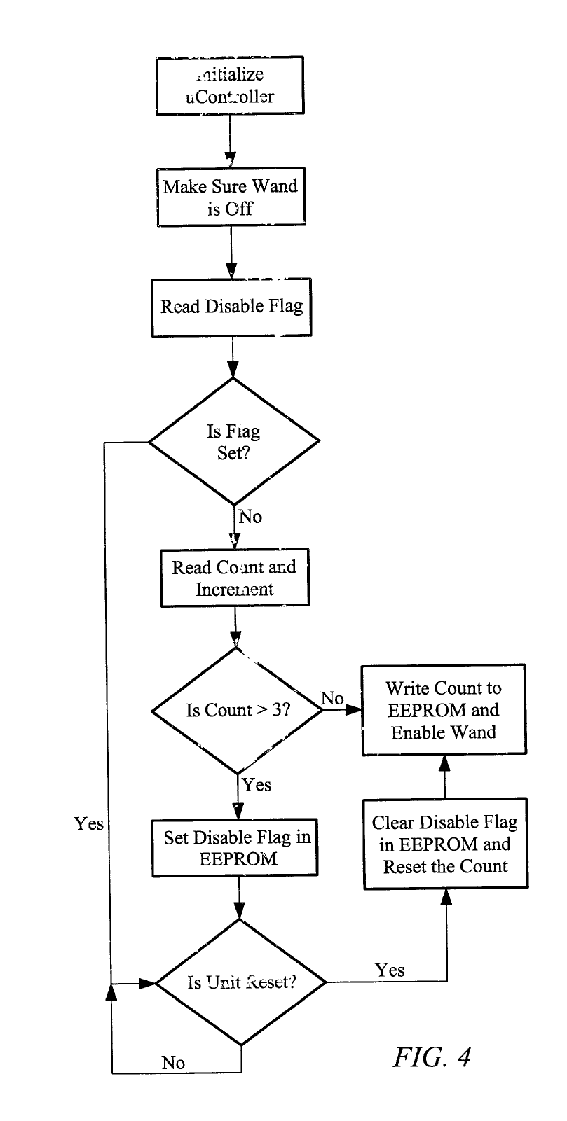

Referring now to FIG. 4, when the limited-use device or, in this instance, the device 10 is energized, the control module 30 initially reads the aforementioned utilization limits and the accumulated utilization history stored in memory module 32. The processing module 40 then reads the “disable flag” to determine if the flag has been set. If the flag has been set, the processing module 40 will further asses if the unit has been reset. If the unit has not been reset, the processing module 40 will not activate the device.

If the flag has not been set, the control module 30 compares each utilization limit to its corresponding value in the accumulated utilization history. For example, in the illustrated embodiment, the limited-use device has an actuation count limit of “3” actuations. The control module 30 will thus compare the actuation count limit of “3” to the total actuation count that has been previously accumulated from prior procedures and stored in the memory module 32 (i.e., EEPROM). If the total actuation count equals or exceeds “3”, the control module 30 will set the disable flag. Obviously, the number of utilization limits is not limited to three but may be selected as desired.

Similar programming may be used to determine if the accumulated actuation time exceeds the corresponding utilization limit. It may also be desirable to program the control module 30 to perform more complicated comparisons between the utilization limits and their corresponding values in the accumulated utilization history. Since the accumulated equipment actuation count and the accumulated actuation time may combine to contribute to equipment deterioration more rapidly than either parameter individually, algorithm utilizing both values may be programmed into control module 30 and used to disable the device.

Additional utilization limits may be programmed and used to disable the limited-use device. For example, chronological time, independent of actual equipment utilization, may contribute to equipment deterioration. It would be a straightforward application of the principles of the present invention to program the memory module 32 with a date of manufacture, or maintenance service dates, and to provide the control module 30 with an internal clock/calendar. The manufacture and maintenance service dates may then be read by control module 30 as previously described, and compared to the clock/calendar. In accordance with the principles of the present invention, a manufacturer may also program and use additional utilization limits that are appropriate for particular medical equipment.

The above-described methods for enabling a limited-use device, such as the RF ablation device 10 illustrated in FIGS. 1 and 2, when utilization limits have been exceeded employ two values for each limit—a preprogrammed utilization limit and an accumulated utilization count. However, in order to conserve memory, in additional embodiments of the invention, only one value for each utilization limit is stored and each preprogrammed utilization limit is decremented as the device is used. For example, the first time a limited-use device is used, the original, preprogrammed utilization limits will be stored in the memory module 32. The control module 30 then decrements each preprogrammed limit during each use. The memory module 32 thus maintains information related to available use remaining for a respective device, instead of utilization limits and accumulated utilization counts.

In accordance with this method, the control module 30 may be programmed to read the available use values immediately after the device is initialized. If an available use value has reached zero, the control module 30 disables the device, thereby preventing further use. Alternatively, the control module 30 may be programmed to perform a more complicated computation using some or all of the available use values to determine if a respective device should be disabled.

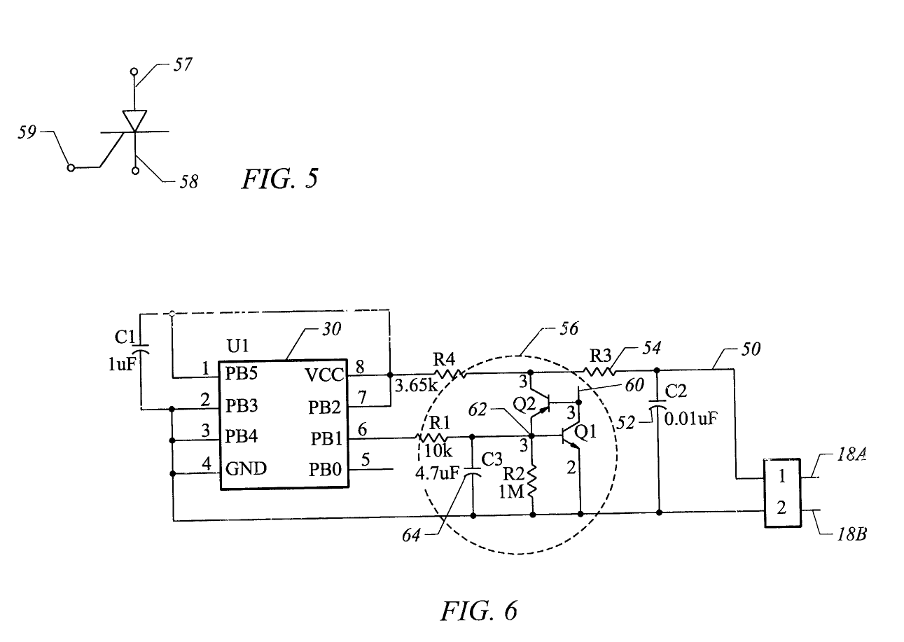

Referring now to FIGS. 5-7, a variation of an enabling means of present the invention will now be described. As illustrated in FIG. 6, the enabling means includes the control module 30, discussed in detail above, and an enabling circuit system 50.

The circuit system 50 is preferably connected to the leads 18a, 18b. According to the invention, the switch 52 and resistor 54 perform the same function as the original identity component 20 (e.g., a voltage drop).

A key feature of the enabling circuit system 50 is the effective silicon control rectifier (SCR) or polychromic switch, denoted generally 56. As will be appreciated by one having ordinary skill in the art, a conventional SCR includes an anode 57, a cathode 58 and a gate 59 (see FIG. 5). In operation, the SCR prohibits current flow until a small positive pulse is applied to the gate 59, producing what is commonly referred to as an “avalanche”, i.e., allows current to flow until current input goes to zero.

The same principle is employed by the effective SCR of the invention. Referring to FIG. 6, a positive potential is exhibited proximate junction 60, which is forward biased (i.e., PN junction). At junction 62 a positive potential is also exhibited. However, in this instance, it is reversed (i.e., NP junction). Thus, current will not be allowed to pass through the system until a positive potential is provided.

In operation, when the limited-use device (e.g., device 10) is initialized, power is provided to the control module 30 at point VCC. The power provided to the system, although insufficient to allow the limited-use device to perform its intended function, is sufficient to power-up the control module 30.

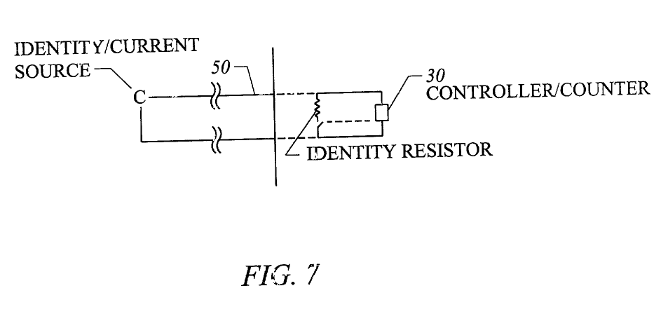

The control module 30 then performs at least one of the above-described programming functions using one or more of the preprogrammed utilization limits (e.g., compare actual equipment actuation count to maximum equipment actuation count). If the device is deemed “underutilized”, e.g., actual equipment actuation count less than maximum equipment actuation count, current is allowed to flow at point or pin “PB1.” As the current is raised at pin PB1, switch 64 ultimately turns on and allows current flow through the system. The noted circuit system 50 is also schematically shown in FIG. 7.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A limited-use device for use with a power supply comprising,
a device body having a proximal and distal portions;
at least one active component located on the distal portion, the component adapted to be activated by the power supply;
a control module attached to the device body, the module adapted to store and compare at least one utilization factor and a utilization history, and being adapted to provide an enablement signal upon comparing the utilization factor and utilization history;
an enablement circuit in communication between the device and the power supply, the enablement circuit including an effective SCR that is adapted to enable the device in response to the enablement current.

2. A limited-use device for use with a power supply, the power supply having an energized and de-energized state, the device comprising,
a device body having a proximal and distal portions;
at least one active component located on the distal portion, the component adapted to be activated by the power supply;
a control module attached to the device body, the module adapted to register a utilization history based on connection of the device to the power supply when the power supply is in the energized state, the control module also adapted to provide an enablement signal upon comparing a utilization factor to the utilization history;
an enablement circuit in communication between the device and the power supply, and adapted to enable the device in response to the enablement current.

3. The limited-use device of claim 1 or 2, wherein the control module comprises a memory module and a processing module, where the processing module and memory module are in communication.

4. The limited-use device of claim 3, wherein the memory module is selected from a device selected from the group consisting of electrically erasable programmable read only memory, non-volatile random access memory, battery backed up random access memory, magnetic data storage apparatus, and optical data storage.

5. The limited-use device of claim 3, wherein the utilization factor and the utilization history are stored in the memory module.

6. The limited-use device of claim 3, wherein the processing module is adapted for comparing the utilization factor and the utilization history.

7. A control system for a limited-use device, comprising:
a control module, said control module having a memory module and a processing module, said memory module being adapted to store an execution program, a plurality of utilization factors and utilization history, said processing module being adapted to monitor said utilization history and provide an enablement current in response to said plurality of utilization factors; and
an enablement circuit, said enablement circuit including an effective SCR that is adapted to enable said device in response to said enablement current.
